(19) United States
(12) Patent Application Publication
(10) Pub. No.: US 2017/0265443 A1
(43) Pub. Date: Sep. 21, 2017

(54) Insect Harborage and Feeding System

(71) Applicant: Delta Five, LLC, Raleigh, NC (US)

(72) Inventors: Robert W. Winston, III, Raleigh, NC (US); Jason A. Janét, Raleigh, NC (US); Stephen P. Land, Raleigh, NC (US); Richard E. Hudson, Raleigh, NC (US); Jesse T. Dean, Willow Springs, NC (US)

(21) Appl. No.: 15/465,244

(22) Filed: Mar. 21, 2017

Related U.S. Application Data
(60) Provisional application No. 62/310,846, filed on Mar. 21, 2016.

(51) Int. Cl. A01K 67/033; B65D 85/50; E04B 1/72; F25D 25/04; A01M 1/02; A01M 1/10

(52) U.S. Cl. CPC A01K 67/033; A01M 1/023; A01M 1/103; E04B 1/72; F25D 25/04; B65D 85/50

(57) ABSTRACT

A harborage for growing and harvesting insects includes a substrate and a mesh material. The substrate has first and second edges extending along the length of the substrate and separated by a shorter width. The substrate includes an absorbent material and is formed into at least one channel extending in a direction along the length of the substrate, in some cases such that the channel is perpendicular to the width and has a depth that is less than the width of the substrate. The mesh material is disposed on the substrate, extends along substantially all of the length of the substrate and includes openings that are smaller than juveniles of a target insect, but sufficiently large to permit liquid feeding of the target insect through the mesh material, which is affixed along the substrate so as to prevent insect egress.

### RELATED APPLICATIONS

This application claims priority from U.S. Provisional Application No. 62/310,846, filed Mar. 21, 2016, the entire contents of which are incorporated herein by reference.

### TECHNICAL FIELD

The present invention generally relates to the production of insect lures, and particularly relates to the use of harborage with a substrate and mesh in a system for harboring, feeding and harvesting insects for lure production.

### BACKGROUND

Lures are employed by pest control entities to draw pests toward a trap or observation device. For example, meal lures appeal to a pest's appetite, while mating lures appeal to a pest's desire to mate. Harborage lures appeal to a pest's desire to find a safe space in which to reside and/or find a mate.

Lures may depend on the state of a pest. Females may be more attracted to a meal lure than a mating lure. On the other hand, males may be more attracted to a mating lure than a meal lure. Pests may be attracted to harborage lures at specific times in their circadian rhythms. Some of these lure attractions/times may be mutually exclusive.

Lures may be natural, synthetic or a blend of natural and synthetic. Synthetic lures are generally comprised of volatile chemical compounds, sometimes referred to as VOC's. Volatiles become gaseous in air, which facilitates their dispersal. Further, many VOC's are considered carcinogenic. In addition, synthetic lures seem to capture only a fraction of the elements that attract pests. Synthetic lures also yield by-products, including offensive odors.

Natural lures are produced directly by mammals, insects, plants, reptiles, birds, etc., and are harvested by a number of methods. One method may be tactile, which includes contact with surfaces that absorb chemicals, such as pheromones, directly from the source. Headspace vapors, which may also include pheromones, emanate from the source and are captured on absorbent materials within that space. Solids, gases and/or liquids that are produced, including feces, urine, saliva, etc., may be captured by absorbent materials. Solids, gases and/or liquids may also be extracted from the source by exposing and/or removing components, including by way of gland removal, digestive tract removal, grinding of body parts, etc. Natural lures may also be comprised of volatile chemical compounds.

Synthetic compounds can be blended with natural compounds to enhance or preserve the lure and/or compensate for missing elements. Natural pheromones generally perform better than synthetic lures. In fact, research literature and empirical testing reveals that synthetic lures do not perform as well as naturally-occurring lures.

Despite their poorer performance, synthetic lures are typically the preferred approach. Synthetic lures are often less expensive to produce than natural lures, because they are typically comprised of a combination of commercially-available chemicals. Synthetic lures are also better known to regulatory entities. Furthermore, handling and caring for live creatures, especially large numbers of live creatures, is considered highly complex and risky.

### SUMMARY

Embodiments of the present invention provide for a more effective lure for insects, such as bedbugs, where the lure includes natural components that are harvested using an efficient and scalable system that handles massive numbers of live insects.

According to some embodiments, a harborage for growing and harvesting insects includes a substrate and a mesh material. The substrate, when configured for growing and harvesting insects, has a width and a length and first and second edges extending along the length and separated by the width, where the length is more than an order of magnitude greater than the width. The substrate comprises absorbent material and is formed into at least one channel extending in a direction along the length of the substrate. The channel may have a depth that is substantially less than the width of the substrate, in some embodiments. The mesh material is disposed on the substrate and extends along substantially all of the length of the substrate. The mesh material comprises openings that are smaller than juveniles of a target insect for the harborage but that are sufficiently large to permit liquid feeding of the target insect through the mesh material. The mesh material is affixed along the substrate so as to prevent egress of insects residing between the mesh material and the substrate.

The substrate may be formed into two or more channels extending along the length of the substrate, and perpendicular to the width of the substrate, in some embodiments. First and second channels of the two or more channels may be separated by a rib between the first and second channels and extending along the length of the substrate. The mesh material may be affixed to the rib in at least an intermittent manner along the length of the substrate.

In some embodiments, the substrate, when configured for growing and harvesting insects, comprises two or more channels extending diagonally along and across the substrate, wherein each of the two or more channels is divided into a plurality of cells.

According to some embodiments, a harborage producing system includes this harborage disposed along a conveyor system and a plurality of insects disposed in and along the at least one channel of the harborage, between the substrate and the mesh material. The harborage producing system may be made up of a number of zones along the conveyor system for handling different steps in the production process. The harborage producing system may utilize various components, such as rollers, a feeder, a freezer, a cutter, a grinder, and/or a shredder.

Of course, the present invention is not limited to the above features and advantages. Indeed, those skilled in the art will recognize additional features and advantages upon reading the following detailed description, and upon viewing the accompanying drawings.

### BRIEF DESCRIPTION OF THE DRAWINGS

FIG. 1A illustrates a sectioned view of the harborage substrate in its completely expanded state, according to some embodiments.

FIG. 1B illustrates a sectioned view of the harborage substrate in a partially expanded state, according to some embodiments.

FIG. 1C illustrates a sectioned view of the harborage substrate in its completely flattened state, according to some embodiments.

FIG. 2A illustrates a sectioned view of the harborage substrate including additional internal harborage material in its completely expanded state, according to some embodiments.

FIG. 2B illustrates a sectioned view of the harborage substrate including additional internal harborage material in a partially expanded state, according to some embodiments.

FIG. 2C illustrates a sectioned view of the harborage substrate including additional internal harborage material in its completely flattened state, according to some embodiments.

FIG. 3 illustrates the different phases of the harborage production process, according to some embodiments.

FIG. 4 shows a typical lab feeder with water, membrane, and blood contents.

FIG. 5 illustrates how a bed bug in a screen-topped lab jar feeds on a typical lab feeder.

FIG. 6 illustrates a configuration of an insect feeder, according to some embodiments.

FIG. 7 is a cross section of the insect feeder showing its blood contents, according to some embodiments.

FIG. 8 is a cross section of the insect feeder with additional heating pipes, according to some embodiments.

FIG. 9 is a cross section of the insect feeder with a blood-displacing insert, according to some embodiments.

FIG. 10 is an example of an open topside trough configuration of an insect feeder, according to some embodiments.

FIG. 11 is an example of a continuous trough membrane configuration of an insect feeder, according to some embodiments.

FIG. 12 is another example of a continuous trough membrane configuration of an insect feeder, according to some embodiments.

FIG. 13 is partially transparent illustration of a continuous trough membrane configuration of an insect feeder showing a blood-heating pipe element, according to some embodiments.

FIG. 14 is a section view illustrating how an insect feeder can be placed laterally across the harborage, according to some embodiments.

FIG. 15 is a section view illustrating an example of multiple insect feeders being used simultaneously,according to some embodiments.

FIG. 16 illustrates another example of a harborage, in this case comprising a plurality of cells disposed along channels extending in a diagonal direction across the substrate/

FIG. 17 is another view of the harborage of FIG. 16.

FIG. 18 illustrates another example of an insect feeder.

FIG. 19 provides a cross-sectional view of the insect feeder of FIG. 18.

### DETAILED DESCRIPTION

It has been observed that bed bugs, cockroaches, etc., rely heavily on pheromones. Pheromones can suggest that a space is safe from predators, that a space is near meal hosts, or that mating opportunities may be nearby. It has also been observed that these pheromones are produced in multiple ways, including secretions, excretions or other gaseous or solid emissions. It has also been observed that these pheromones are transferred to surrounding/nearby materials and/or environment in multiple ways. These ways may be tactile, i.e., involving physical contact, or via the absorption of vapors into headspace.

It has further been observed that adult female bedbugs (and possibly adult females of other insect types) may venture away from colonies to find refuge, whereas adult males often stay close to a base harborage. It has also been observed that adult egg-laying females may seek and take more meals than males due to their reproductive needs.

Consideration has been given to the need to lure bed bugs to a trap or sensor. One example sensor that may effectively use a lure is the automated insect monitoring system described in U.S. Patent Application Publication US 2016/02387347, the entire contents of which are incorporated herein by reference. However, to date there has been a lack of effective, commercial, off-the-shelf, lures. Synthetic lures are available, but they generally underperform relative to natural lures. Synthetic lures typically have inherent risks as well, such as offensive odors, VOC's or flammability.

Embodiments of the present invention, as detailed herein, provide for a more effective lure with natural components that are harvested from insects using a scalable system that handles massive numbers of live insects. The system is a cost-effective means of growing and harvesting a natural lure. This approach is applicable to bed bugs, but the various techniques and equipment described herein may also be used to derive natural lures from other pests including roaches, weevils, moths, etc.

Features of the techniques and systems described herein include a continuous harborage, suitable for large-scale manufacturing, that is to conducive to comfortable habitation, breeding and feeding. This harborage is fully enclosed and is used to maximize production of lure components by feeding and keeping colonies of insects healthy and productive, while maintaining environmental conditions to support rapid maturity. This approach also maximizes the harvesting of lure components. The substrate of this harborage is comprised of materials that capture pheromones, vapors, liquids, solids, etc., that can he harvested, packaged, and distributed for use as lures. Note that the term “substrate” as used herein is meant in the sense of a material on which a process occurs, in this case the process of growing insects and collecting the pheromones, vapors, liquids, that result from this process. Containment materials (e.g., plankton netting mesh) that form a portion of this harborage may also be capable of capturing lure components, and can also be harvested. Exuvia (cast-off outer skin), recently deceased insects, and other detached solids that remain in the harborage can also be harvested and/or integrated into the lure.

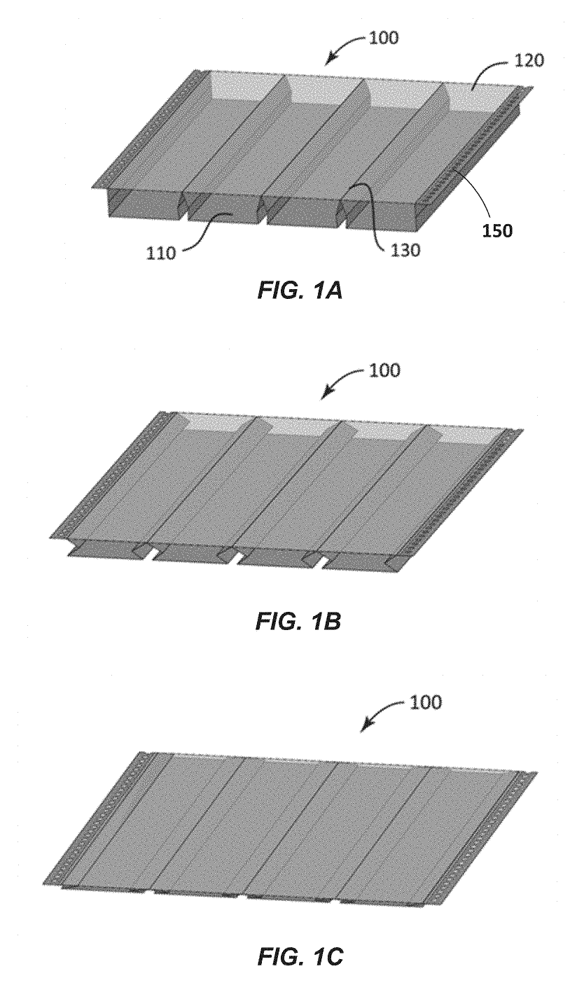

The harborage, in some embodiments, comprises a substrate made of absorbent material, such as paper or cloth, or a mixture thereof. FIGS. 1A-1C show a substrate 150 of an example harborage 100, where the substrate 150 is shaped into one or more channels or gutter-like lanes 110. The substrate 150 may also include a floor, gutter side walls and a continuous perimeter to prevent bedbugs within the harborage from escaping. The gutter walls may be pleated to fold into a thin profile, as shown in FIG. 1B. The substrate 150 may also be flattened, as shown in FIG. 1C. This can facilitate storage, including rolling. The substrate 150 may be configured to facilitate pinch-based boundaries.

In the pictured embodiment, the top, or ceiling or roof, of the harborage may be made of a mesh 120. Some sides may also be made of the mesh 120. The substrate 150 may include openings that are sized to be smaller than the smallest resident of the substrate 150. The openings may also be sized to facilitate feeding. The, openings may be sized to permit atmospheric equalization, to prevent trapped humidity that might cause mold or other problems.

The mesh 120 may be adhered to the substrate 150 completely along the perimeter to prevent escape. The mesh 120 may also be adhered completely or intermittently along ribs 130. The substrate 150 may be configured to keep the ribs 130 aligned and integrated with the mesh 120. The substrate 150 may also be configured to facilitate movement of bugs between lanes 110, to equalize the distribution of bugs. For example, in some embodiments, there may be intermittent openings in the ribs 130, to allow insects to move from one channel or lane 110 to another.

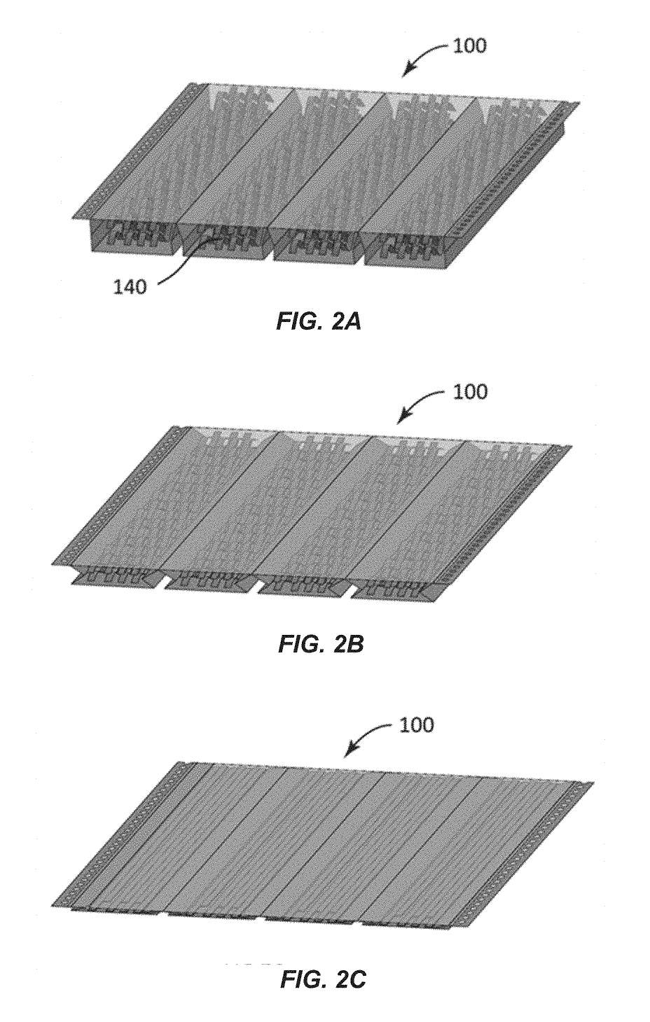

The internal volume of the substrate 150 may include additional harborage material 140, as shown in FIGS. 2A to 2C. This material 140 may include absorbent material that is any combination of: shredded, folded, baffled, segmented, crumpled, die-cut or honeycomb. The additional harborage material 140 may be configured to maximize the density of harborage for bugs and/or to maximize the density of pheromone-infused substrate. As shown by FIGS. 2A-2C, the additional harborage material 140 may be designed so that the substrate 150 may still fold into a thin profile or be flattened.

According to some embodiments, a harborage for growing and harvesting insects to form insect lures, such as harborage 100, includes a substrate and a mesh material, such as substrate 150 and mesh 120. The substrate, when configured for growing and harvesting insects (i.e., when not in a flattened or folded configuration prior to use), has a width and a length and first and second edges extending along the length and separated by the width, where the length is more than an order of magnitude greater than the width. The substrate comprises absorbent material and is formed into at least one channel extending in a direction along the length of the substrate. The channel may have a depth that is less than the width of the substrate, for example. The mesh material is disposed on the substrate and extends along substantially all of the length of the substrate. The mesh material comprises openings that are smaller than juveniles of a target insect for the harborage but that are sufficiently large to permit liquid feeding of the target insect through the mesh material. The mesh material is affixed along the substrate, e.g., in a substantially continuous manner, so as to prevent egress of insects residing between the mesh material and the substrate.

The substrate may be formed into two or more channels extending along the length of the substrate, and perpendicular o the width of the substrate, in some embodiments. First and second channels of the two or more channels may be separated by a rib between the first and second channels and extending along the length of the substrate. The mesh material may be affixed to the rib in at least an intermittent manner along the length of the substrate.

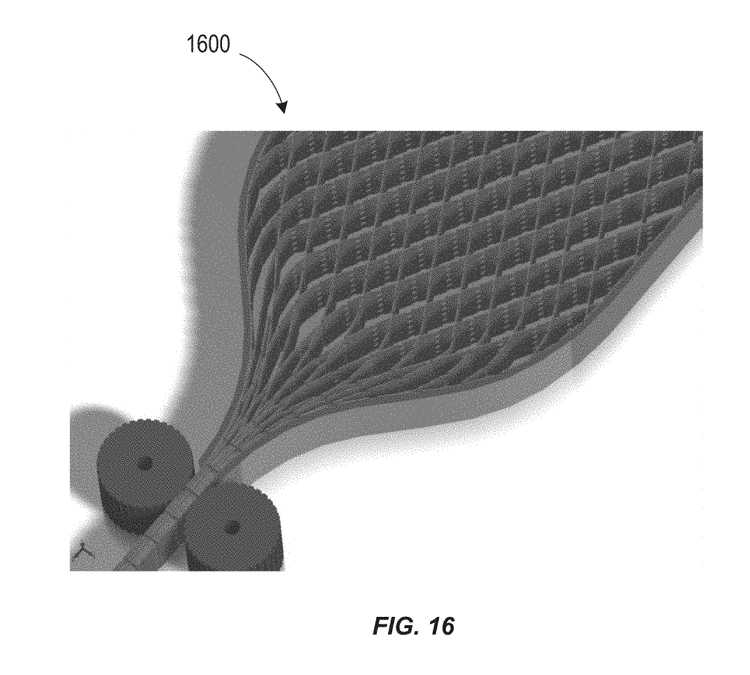

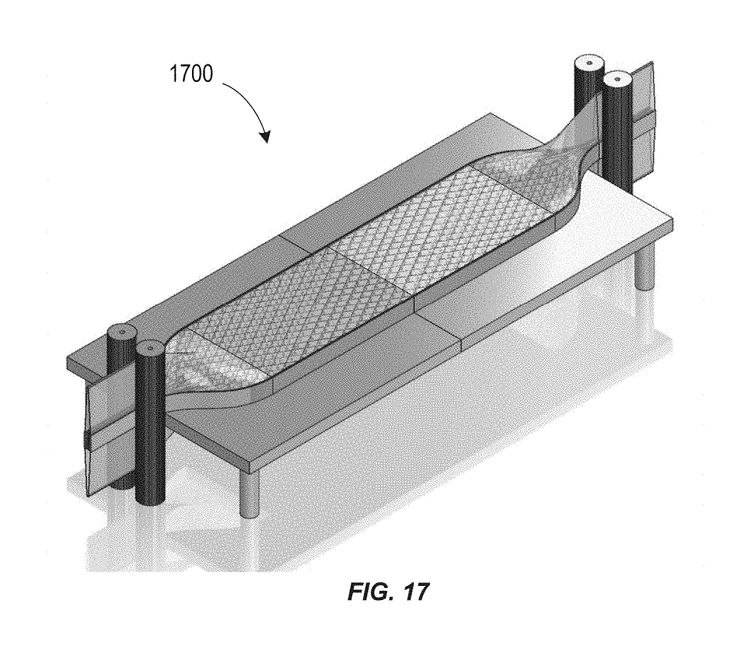

In some embodiments, the substrate, when configured for growing and harvesting insects, comprises two or more channels extending diagonally along and across the substrate, wherein each of the two or more channels is divided into a plurality of cells. An example of such an embodiment is shown in FIGS. 16 and 17. The cells in harborage 1600 can be seen in both figures; FIG. 17 illustrates a harborage system 1700 in which the harborage may be deployed from a flattened position through the use of spreading rollers, e.g., at the left side of the figure, and then restored to a flattened position at the other end of the feeding and growing zone, using another pair of rollers.

The harborage may include additional absorbent harborage material disposed in and along the at least one channel, the additional harborage material comprising one or more of: shredded material; folded material; baffled material; segmented material; crumpled material; die-cut material; and honeycomb material.

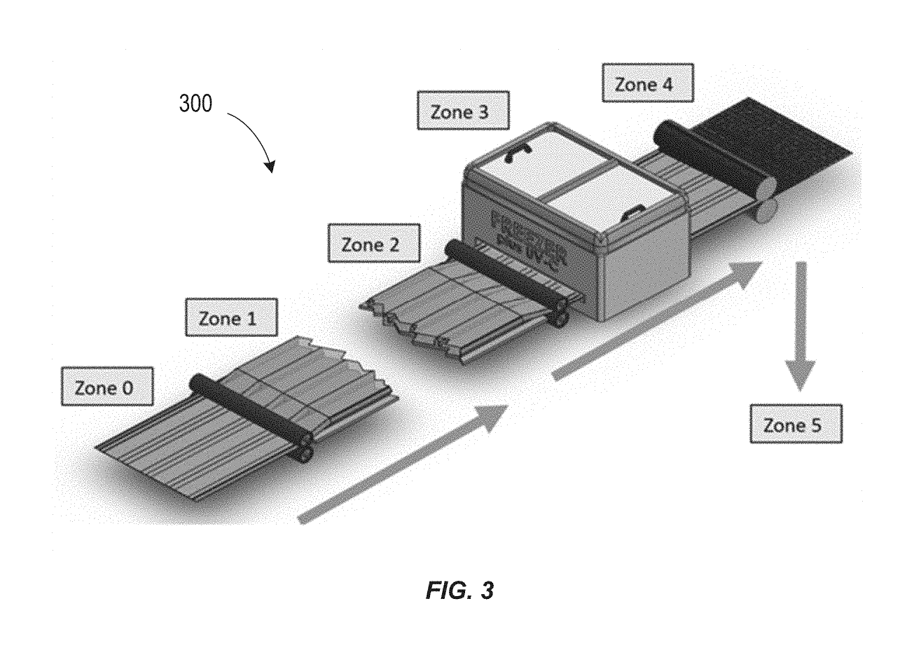

FIG. 3 illustrates an embodiment of a harborage producing system 300 with a continuous harborage. Different phases of the harborage production process are shown by Zones 1-5 of the harborage producing system 300. The continuous harborage producing system 300 is a conveyor system that both contains and transports colonies of bugs and their pheromones through the multiple zones. Zone 0 is a pre-harborage zone. This zone includes storage of harborage material. In this zone, the substrate 150 is preferably compact to facilitate efficient storage, possibly on a roll. One or more compliant rollers compress the harborage material at a point or region of the harborage 100, to form a boundary and prevent passage of pests from Zone 1 into Zone 0.

Zone 1 is a harborage zone. This may be a climate-controlled region, in some embodiments. This zone may contain a large collection of bugs and the pheromones they produce. Bugs can age, feed, procreate and dwell comfortably in this zone. The length of Zone 1 is adaptable and, preferably, long enough, when the movement of the harborage is accounted for, to allow bugs to grow to adulthood and propagate, even if they hatch mid-way along the zone. Note that the feeding regimen may be designed to draw bugs closer to Zone 0. This can concentrate more meals upstream and maximize feeding frequency upstream. Bed bugs can move about 25 feet per hour—this speed of movement should be factored into the design of the system, i.e., with respect to the length of Zone 1 and the speed at which the :harborage moves through the system 300.

Zone 2 is a stress region. It is a climate-controlled region, where bugs can age, procreate and harbor. Bugs are encouraged to leave this zone to move back to Zone 1. Bugs are not fed in this zone. Bugs may be repelled in this zone (e.g., cold temperatures, sounds, scents, etc.). Most likely only the fittest bugs will move upstream to Zone 1, where they can be fed. Weak, unfortunate, and dead bugs will remain in this zone, however.

Zone 3 is a neutralization region, where harborage and its contents (e.g., bugs, pheromone-infused substrate, and containment material) move into a freezer. Additional mechanisms may be employed, including UV light, pesticide, dehumidification, etc. The freezer also stores neutralized harborage in a manner that minimizes degradation.

Zone 4 is a bulk process lure mix ref-lion. This may include mechanical operations, such as grinding, shredding, cutting, etc. This may also include chemical operations, such as extracting, embellishing, etc. The lure mix is treated to minimize the likelihood of problems for the end-user (e.g., mold).

Zone 5 is a region for packing, storing and shipping. This may include anything from bulk packaging to unit-dose packaging. The product is placed in appropriate storage and shipped in appropriate containers to its destination.

According to some embodiments, a harborage producing system, such as system 300, is disposed along a conveyor system and includes a plurality of insects disposed in and along the harborage, e.g., along at least one channel of the harborage, between the substrate and the mesh material. The harborage producing system also includes one or more rollers disposed across the harborage at a first end of a first zone extending along the harborage, where the one or more rollers are disposed so as to compress the harborage and thereby form a boundary preventing the live passage of insects past the first end of the first zone.

The harborage producing system may include one or more additional rollers disposed across the harborage at a second end of a first zone extending along the harborage, the second end of the first zone being separated from the first end of the first zone by a first zone length. The one or additional rollers are disposed so as to compress the harborage and thereby form a boundary preventing the live passage of insects past the second end of the first zone.

The harborage producing system may further include a freezer, where the harborage and conveyor system are disposed to pass the harborage through the freezer. The harborage producing system may also include a cutter, grinder, and/or shredder, the harborage and conveyor s being disposed to pass the harborage into the cutter, grinder, and/or shredder.

Insects that feed on blood use a feeding appendage, such as a proboscis, to penetrate one or more membranes. Many insects inject a substance that conditions the blood and/or desensitizes the flesh near the insertion point to not alert animal. The proboscis acts like a straw to draw blood into the insect's digestive system. The state of the flesh, which sometimes doubles as a lure, is warm (for warm-blooded creatures), typically 97° to 100° F. Flesh may emit a flesh-like scent, which is alluring to blood-eating insects. Flesh may also pulsate, such as from a beating heart, which may also be alluring or facilitate orientation.

Most lab-based approaches feed discrete, small populations. The feeders may be clear, cup-like containers with mesh on top and folded paper inside. The paper enables bed bugs to climb and harbor. The mesh contains the bugs, but allows a proboscis to penetrate for feeding. These containers typically house less than 2000 bugs, and this density is too small for producing lures. But, more importantly, this approach makes it very difficult to harvest the pheromone-infused substrate without killing the vast majority of the colony and laboriously separating live bugs, many barely visible to the naked eve,from the substrate. This approach also risks the escape of bugs.

In contrast, some of the embodiments described herein are intended to let only dead, lethargic, and old/weak bugs get compromised, when processed after Zone 2. Since males seem less prone venture to find new harborages, males may have a greater tendency to be compromised during the harvesting process. However, a colony needs only a small or minority fraction of its population to be adult males, and females can lay up to dozens of eggs per insemination

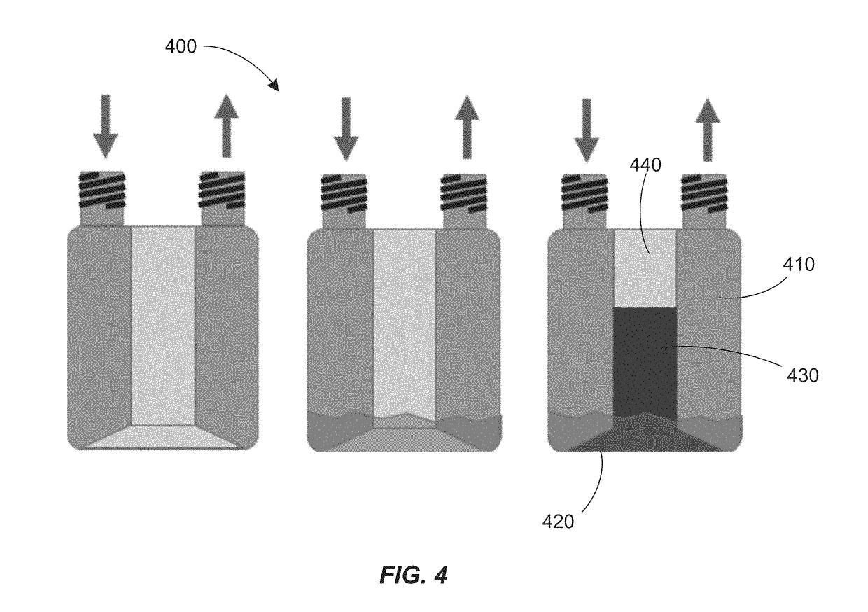

FIG. 4 shows a typical lab feeder 400 with water 410, membrane 420 and blood 430 contents. Typical lab feeders involve a low number of bugs (hundreds to tens of thousands). Some labs use humans or animals to feed bugs. But most labs use feeder emulators, which include a water heater-circulator to warm feeding vessels, pipes to guide heated water from a heater-circulator to a feeding vessel and a double-wall vessel for blood (or other liquid meal). Heated water circulates between the inside and outer walls. Blood resides in an inner well (“chimney”) 440, which is typically only 1.2 cm in diameter, holding only 6.75 cm³. These feeders 400 often require significant labor to monitor and replenish. The membrane 420 contains the blood 430 in the well using, for example, a synthetic membrane, sausage casings, etc. The membrane 420 may be retained with rubber bands, zip ties, additional membrane layers, etc. The membrane 420 utility is typically less than 120 minutes due to dehydration and/or the solidification of the blood 430. This means that the typical feeders must undergo the laborious task of membrane replacement.

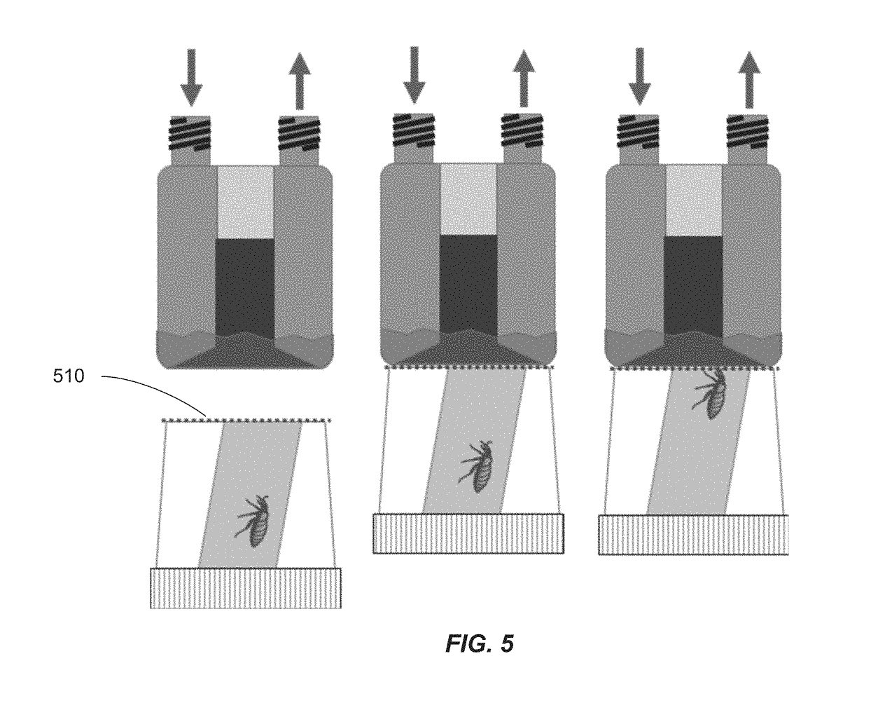

Bugs feed when the warmed blood 430 and membrane 420 contact or are very close to the mesh 510. FIG. 5 illustrates how a bed bug in a screen-topped lab jar feeds on a typical lab feeder 400. The bugs will climb to the mesh 510. The bug's proboscis penetrates he mesh 510 and the membrane 420. The bugs may condition the blood 430 they access and then take blood through the proboscis until satiated. The typical lab feeder area is about 30 cm². This limits the number of bugs that can feed, and most feasible membrane materials limit the feeder area to 30 cm² due to their natural size (e.g., intestine diameter) or manufactured size (e.g., 3M film, NESCO film, etc.).

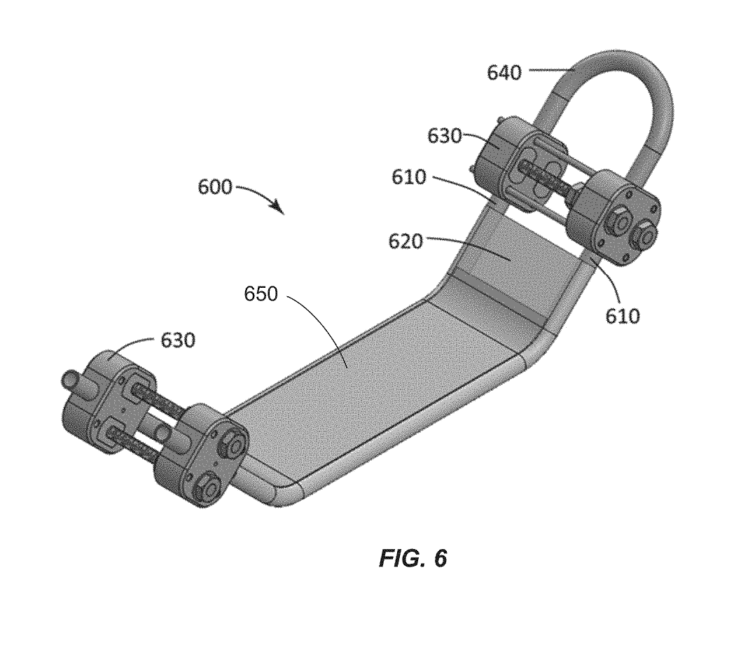

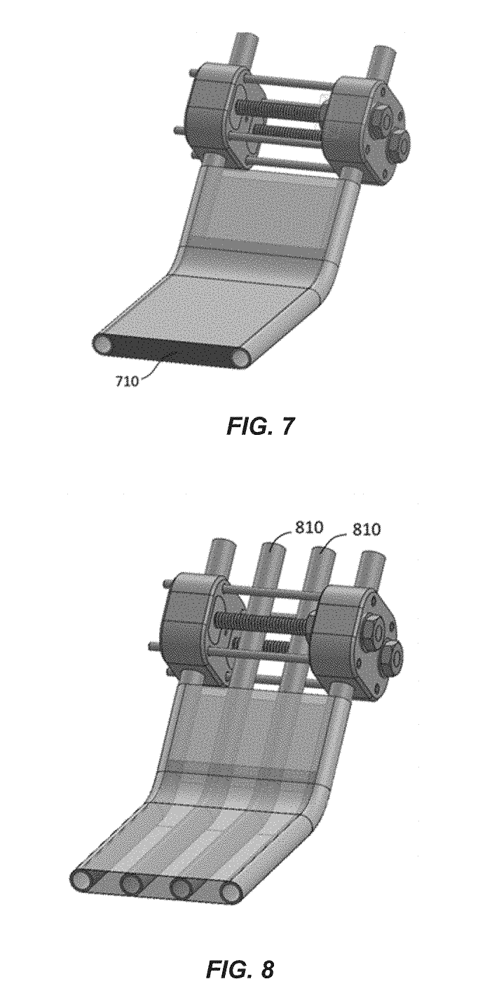

FIG. 6 illustrates a configuration of a tube-based feeding vessel chassis, shown by feeder 600, according to some embodiments. The feeder 600 is compatible with the harborage and harborage producing systems described above. FIG. 7 is a cross section of the feeder 600, showing its blood contents 710. The feeder 600 provides significant scalability in one or more dimension(s), including the feeder area and/or feeder capacity. The feeder 600 enables the feeding of very large populations simultaneously, rapidly and with minimal labor. The feeder 600 also facilitates conservation and the recovery of blood or other liquid meal.

The design of the feeder 600 may include a water heater-circulator to warm one or more feeding vessel. Pipes guide heated water from the heater-circulator to the feeding vessels. The feeder 600 includes chassis that provide structural stability to the feeder 600. The chassis may have at least two chassis portions that are used to spread a membrane 620 taught across a width between the chassis portions. The chassis portions may be solid or may be tubes that are configured to provide heat to warm the blood 710 or other liquid meal. Heated water or other fluid may circulate in such chassis tubes. The chassis portions may be thermally-conductive and/or made of aluminum, glass, etc. Chassis tubes 610 are shown in FIG. 6 and additional heating elements, such as chassis tubes 810 are shown in FIG. 8.

The chassis tubes 610 occupy an interior space within the membrane 620, which may form a membrane tube 650. The length and width of the membrane 620 can scale with the membrane tube's 650 diameter. The chassis tubes 610 may protrude past ends of the membrane tube 650. As shown by FIG. 8, multiple chassis tubes 810 may occupy the interior of the membrane tube 650 to provide sufficient heating elements.

The chassis tubes 610 may be mechanically coupled with one or more couplers 630 that hold both chassis tubes 610 on one or more ends. Each coupler 630 may have a mechanism to spread the chassis tubes 210 in order to vary membrane tightness. A return bend 640 of the chassis tubes 610 may also provide structural integrity at one end. In some cases, only one coupler 130 is used in combination with the return bend 640 or similar structure to vary the membrane tightness.

The membrane 620 forming the membrane tube 650 may be tightened to a preferred tightness by spreading the chassis tubes 610. The blood (or other liquid meal) 710 pools inside the membrane tube 650. The blood 710 is warmed due to contact with the chassis tubes 610, 810. The membrane tube 650 that contains the blood 710 may be formed by a synthetic membrane or a natural membrane, such as a sausage casing. The membrane 620 may be retained at ends with rubber bands, zip ties, clamps, additional membrane layers (e.g., folds), etc. The chassis tubes 610 may have angled or vertical bends to form ends of a blood pool boundary. For example, the membrane tube 650 may bend vertically to contain blood pool at each end. This may allow one or both ends of the membrane tube 650 to be above the blood pool. This may allow the membrane tube 650 to form a reservoir to replenish the blood from the pool that is consumed.

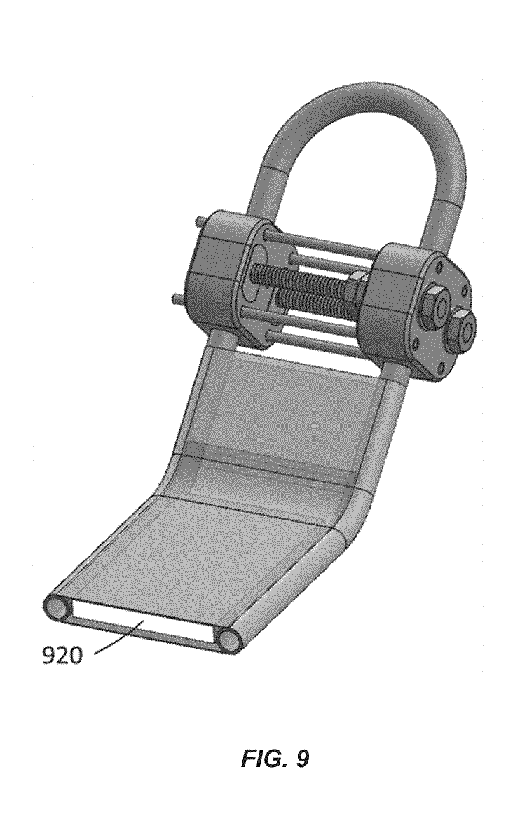

As mentioned above, the membrane utility may be less than 120 minutes, so it s important to simultaneously feed as many bugs as possible. The membrane 620 may suffer from dehydration, solidification of blood along the membrane's 620 wall or floor, settling of suspended solids, or perforations from bug proboscies. To address this issue, a feeder insert may be placed within the membrane tube 650, such as shown by feeder insert 920 in the cross-section of the feeder 600 illustrated by FIG. 9. The feeder insert 920 may be constructed from a closed-cell, buoyant material, so that it rises to contact the top of the membrane tube 650, but leaves distance between the bottom of the membrane tube 650 and the bottom of the feeder insert 920. This distance will help to control the blood pool height to be just above a proboscis, which is the critical height. This will also help to minimize the amount of the blood 710 sitting in the membrane tube 650 and provide thermal insulation on ceiling of the membrane tube 650. The feeder insert 920 may also be a rigid ceiling that connects to the chassis tubes 610.

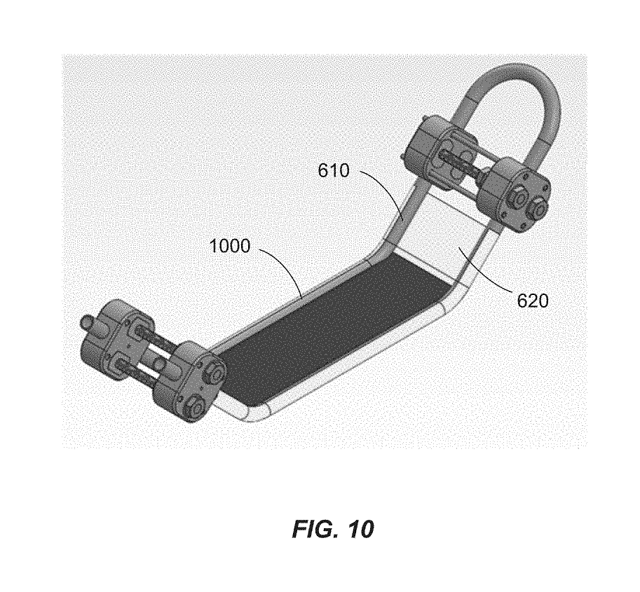

The membrane 620 used to form the membrane tube 650 may also be used to form a membrane trough. This membrane trough may be formed similar o the membrane tube 650. FIG. 10 is an example of an open topside trough configuration of a feeder, according to some embodiments. The membrane trough 1000 of FIG. 10 may be open on the topside but otherwise formed similar to the bottom and sides of the membrane tube 650. For example, the chassis tubes 610 hold and spread the membrane trough's 1000 walls to provide a preferred tightness of the membrane 620 at the bottom of the membrane trough 1000. The blood 710 pools inside the membrane trough 1000 and is warmed due to contact with the chassis tubes 610.

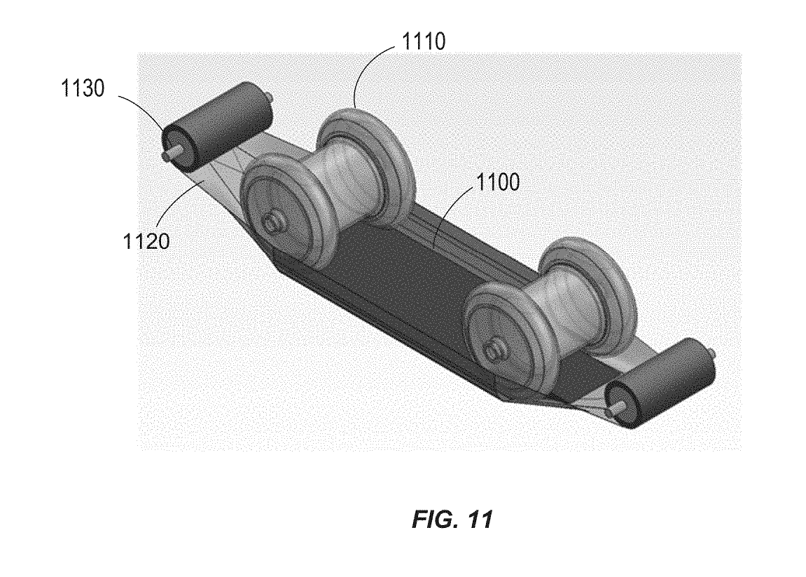

FIG. 11 is an example of a continuous trough membrane configuration of a feeder, according to some embodiments. The continuous membrane trough 1100 formed by the membrane 1120 is open topside and includes rollers 1130 that dispense and recover the membrane 1120. The membrane 1120 continuously refreshes without the need to recover or transfer the blood 710. The membrane 1120 may be made of the same material as described above, and tightened and retained in the same fashion. Chassis tubes 1110 guide and spread the membrane's 1120 walls to provide the trough's 1100 walls and floor at a preferred tightness. The blood 710 pools inside the trough 1100 and is warmed due to contact he chassis tubes 1110.

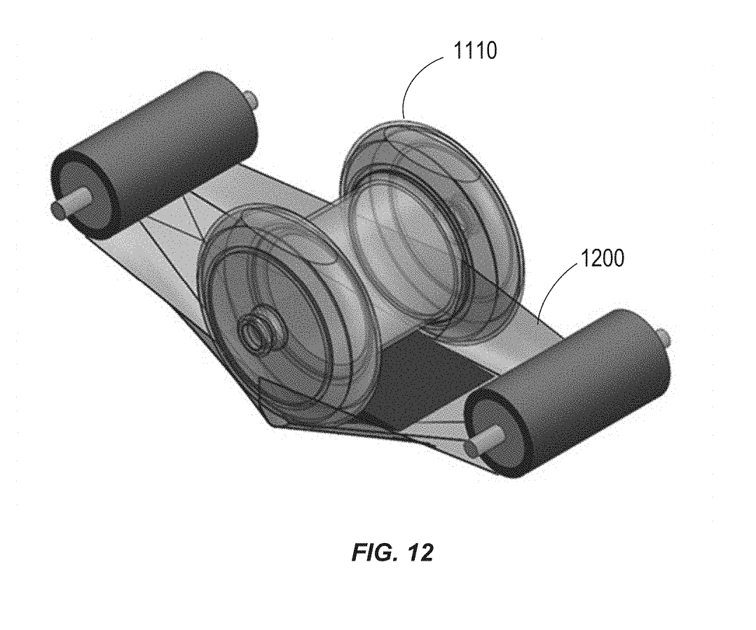

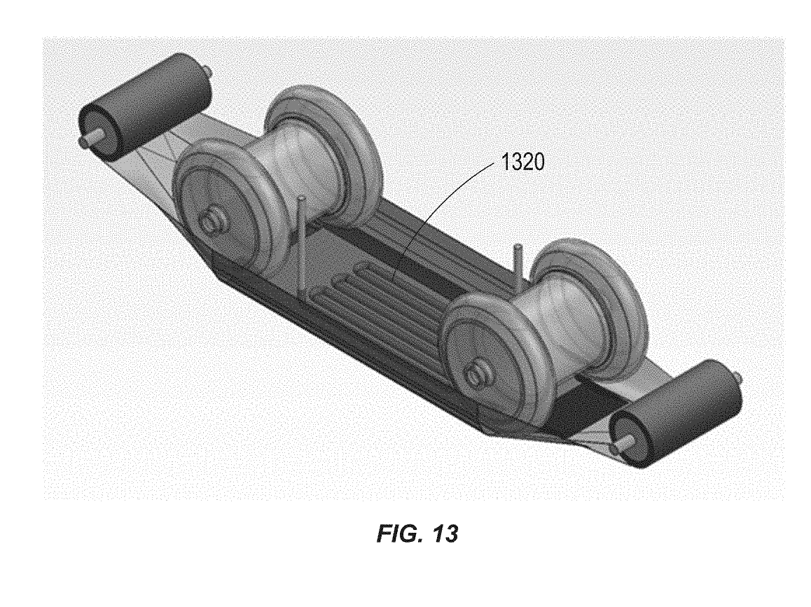

FIG. 12 shows another configuration of a continuous membrane trough 1200 formed by just one roller/set of chassis tubes 1110. FIG. 13 is partially transparent illustration of a continuous trough membrane configuration of an insect feeder showing a blood-heating pipe element 1320, according to some embodiments.

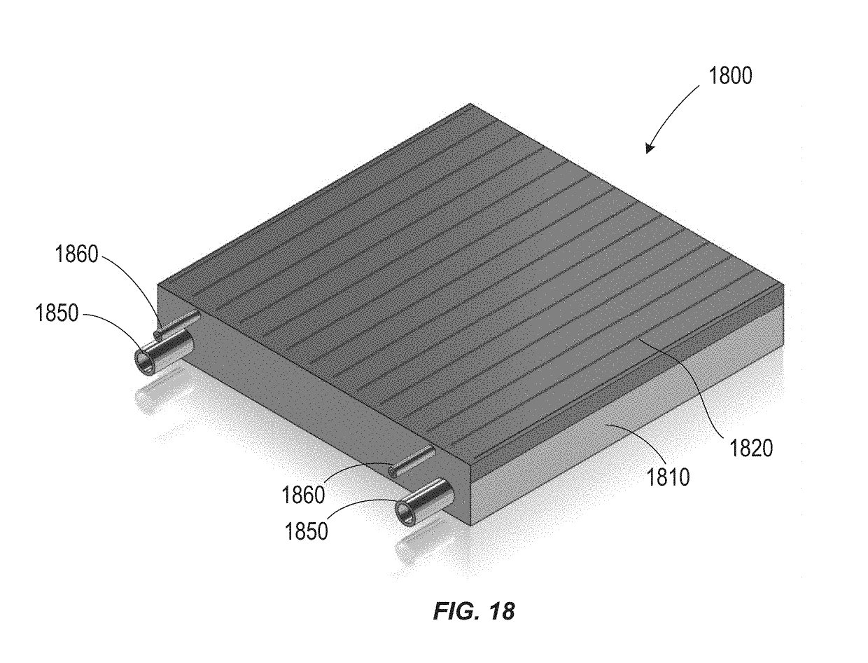

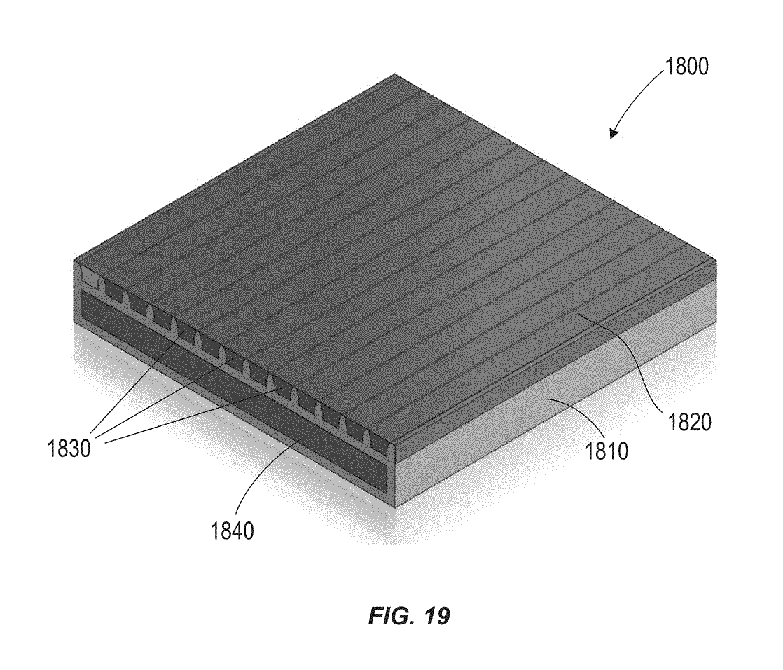

FIG. 18 illustrates another example feeder configuration, with FIG. 19 providing a cut-away view of the same feeder. This embodiment comprises a unique heat exchanger, which positions a feeder membrane across one broad surface of heated volume of blood, while at the same time providing adequate structural and positional support across the span of the membrane. In the pictured embodiment, heat is introduced into the system via a fluid such as warmed water, which may be circulated both to and from an internal reservoir 1840 of the heat exchanger body through inlet and outlet piping 1850. Heat is conductively transferred from this fluid through the interior partition of the heat exchanger body and to the blood food liquid by the opposing face of the partition, aided, in some embodiments, by heat-sink-like finned structures on one or both sides of the partition. Blood may be either captively retained on the membrane side of the heat exchanger feeder, or circulated through this region of the feeder through inlet and exit piping 1860. Note that other embodiments may heat the heat exchanger via embedded internal heating elements such as electrical resistance heaters.

Feeder 1800 thus comprises a feeder body 1810, which may comprise a piece of molded or cast metal, in some embodiments. A feeder membrane 1820 is affixed to a first surface of the feeder 1800. In the illustration, the membrane 1820 is on a top surface of the feeder 1800, although it will be appreciated that the feeder 1800 may typically be oriented with the membrane 1820 down, when feeding insects. Underneath the member 1820 are several channels or reservoirs 1830, which may be filled with blood for feeding the insects.

It will be appreciated that elements of the body 1810 extend between the channels or reservoirs 1830—this facilitates both a limiting of the reservoir area, to avoid some of the problems discussed above, but also allows for improved warming of the blood, as a metallic body 1810 can communicate heat to the blood in the reservoirs more effectively with this arrangement. On the blood retaining side of the conductive partition, these extensions, which may be regarded as “heat fins,” also may provide two additional functionalities. First, these fins may provide positional support for the feeder membrane. The feeder membrane, when oriented as shown in FIGS. 18 and 19, will be supported underneath against gravitational downward forces by the normal force from the heat fins in contact with the membrane. In other positons, including vertical or inverted to the illustrated position, the membrane may be attached to the fins, wholly or at specific point of contact, by features molded into the underside of the membrane (such as nipples or “T-slot” features) which will allow the membrane to remain flat and in contact with the entirety of the support structure.

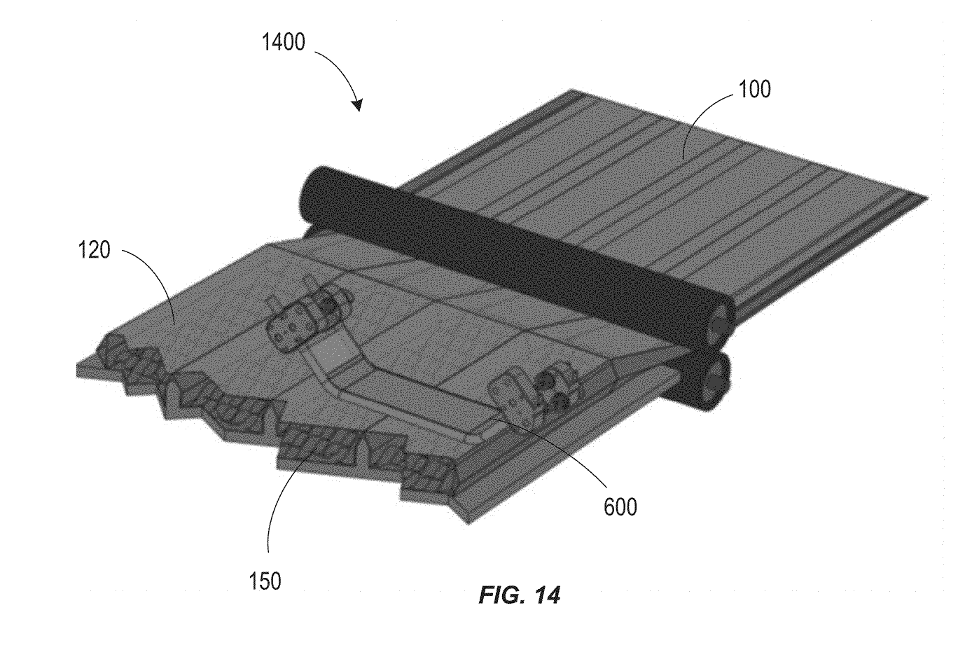

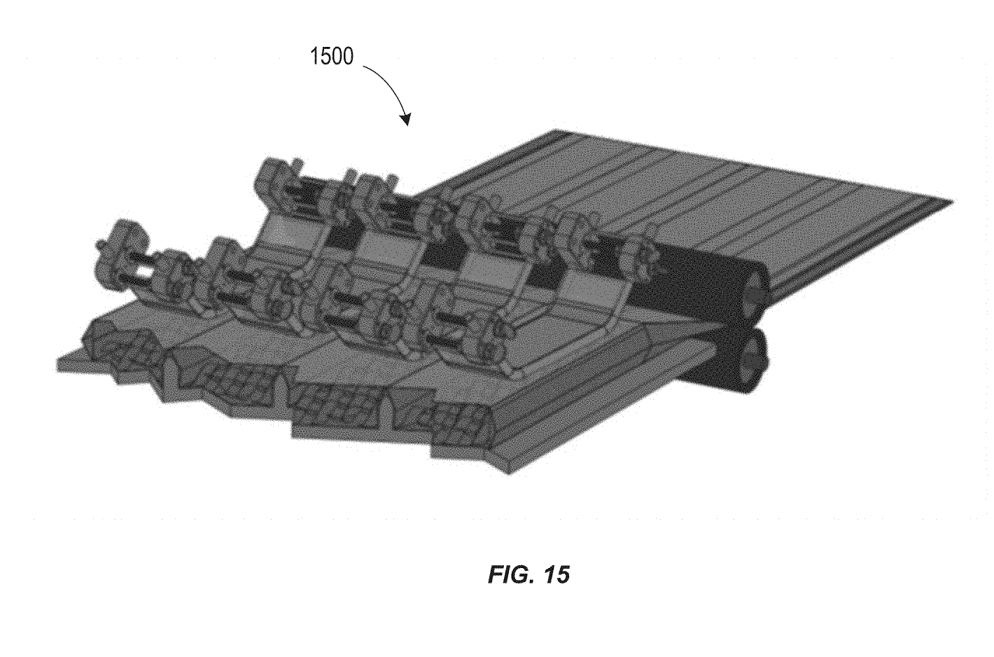

FIG. 14 is a section view illustrating how a feeder can be placed laterally across the harborage, according to some embodiments. For example, the portion of a harborage producing system 1400 shown in FIG. 14 includes a feeder 600 that is in contact with or minimally distanced from the substrate 150 of a continuous harborage 100, such that a proboscis from a bug in the substrate 150 may feed from the blood in the feeder 600 through the mesh 120 of the continuous harborage 100. The feeding vessel membrane of each feeder 600 presses against the mesh 120 of the continuous harborage. 100. The warmed blood (or other liquid) resides above the membrane floor. Bugs feed through the mesh 120 and the membrane floor. Note that while the feeder 600 in FIG. 14 is disposed laterally across the harborage 100, it might alternatively be disposed longitudinally, i.e., along the harborage 100, e.g., as shown in FIG. 15.

FIG. 15 is a section view illustrating an example of multiple feeders 600 being used simultaneously, according to some embodiments. The multiple feeders 600 of system 1500 may be placed anywhere on the continuous harborage 100, including across multiple lanes or generally parallel to the lanes. For example, the chassis tubes 610 of the feeders 600 may straddle or go between the ribs 130 of the substrate 150.

The systems 1400, 1500 may provide significant scalability in one or more dimensions. The feeding area varies with feeding vessel length and membrane tube diameter. A feeding area may be, for example, 4.75 cm×17.75 cm, or 84.3 cm². This is almost four times the area of a typical lab feeder.

However, natural and synthetic sausage casings can come in lengths upwards of 20 meters. Therefore, feeding areas can be extended to yield significantly larger feeding areas. For example, a 4.75 cm×40 cm feeding area would be 190 cm², which is nine times more than a typical lab feeder, and so on. In fact, natural and synthetic sausage casings can come in diameters upwards of 15 cm, resulting in even larger feeding areas.

The feeder capacity varies with feeding vessel length and membrane tube diameter Other factors contribute to the feeder capacity. Ignoring the potential reservoirs at both ends, and without the insert, a feeder 600 may support a capacity volume, for example, of 4.75 cm×17.75 cm×1 cm, or 84.3 cm³. This is about 12.4 times a typical lab feeder. Capacity increases by adding the vertical reservoirs at both ends. A feeder insert may reduce the blood pool capacity. In an example, a feeder insert 920 displaces about ⅔ of the blood pool, resulting in a blood pool capacity of 28.1 cm³. This is about 4.2 times the typical lab feeder. Also, natural and synthetic sausage casings can come in lengths upwards of 20 meters and thus feeder volumes can be extended to yield significantly larger capacities. Natural and synthetic sausage casings can also come in diameters upwards of 15 cm, resulting in even larger feeder volumes.

According to some embodiments, the harborage of a harborage producing system, such as system 1400 or 1500, is disposed along a conveyor system and includes a plurality of insects disposed in and along the at least one channel of the harborage, between the substrate and the mesh material. The harborage producing system may include one or more rollers disposed across the harborage at a first end of a first zone extending along the harborage, where the one or more rollers are disposed so as to compress the harborage and thereby form a boundary preventing the live passage of insects past the first end of the first zone.

The harborage producing system may include one or more additional rollers disposed across the harborage at a second end of a first zone extending along the harborage, the second end of the first zone being separated from the first end of the first zone by a first zone length, wherein the one or more additional rollers are disposed so as to compress the harborage and thereby form a boundary preventing the live passage of insects past the second end of the first zone.

The harborage producing system may further include a freezer, the harborage and conveyor system being disposed to pass the harborage through the freezer. The harborage producing system may also include a cutter, grinder, and/or shredder, the harborage and conveyor system being disposed to pass the harborage into the cutter, grinder, and/or shredder.

Advantages of the described system, include the ability to feed very large populations simultaneously, rapidly and with minimal labor. Zone 1 of a typical infinity harborage may be required to support in excess of one million bed bugs. The feeders 600 are designed to support this requirement. Considering the feeder areas and capacities alone, labor can be reduced by at least a factor of four, and upwards to a factor of twelve or more. These savings continue to increase with longer and/or wider feeders.

Other advantages include the ability to facilitate conservation and recovery of the blood or other liquid meal. The feeder insert minimizes the blood in the pool to what is essential for bugs to feed without damaging their proboscis. The openings at each end of the membrane tube allows new blood or liquid meal to be poured in. Any unused blood or liquid meal may be poured out. The removed liquid may be poured into a new feeding vessel.

Notably, modifications and other embodiments of the disclosed invention(s) will come to mind to one skilled in the art having the benefit of the teachings presented in the foregoing descriptions and the associated drawings. Therefore, it is to be understood that the invention(s) is/are not to be limited to the specific embodiments disclosed and that modifications and other embodiments are intended to be included within the scope of this disclosure. Although specific terms may be employed herein, they are used in a generic and descriptive sense only and not for purposes of limitation.

What is claimed is:

1. A harborage for growing and harvesting insects, the harborage comprising:
a substrate having, when configured for growing and harvesting insects, a width and a length and first and second edges extending along the length and separated by the width, the length being more than an order of magnitude greater than the width, wherein the substrate comprises absorbent material and is formed into at least one channel extending in a direction along the length of the substrate; and
a mesh material disposed on the substrate and extending along substantially all of the length of the substrate, the mesh material comprising openings that are smaller than juveniles of a target insect for the harborage but that are sufficiently large to permit liquid feeding of the target insect through the mesh material, the mesh material being affixed along the substrate so as to prevent egress of insects residing between the mesh material and the substrate.

2. The harborage of claim 1, wherein the at least one channel has a depth that is less than the width of the substrate.

3. The harborage of claim 1, wherein the substrate is formed into two or more channels extending along the length of the substrate and perpendicular to the width of the substrate.

4. The harborage of claim 3, wherein the mesh materially is affixed along each of the first and second edges of the substrate in a substantially continuous manner.

5. The harborage of claim 3, wherein first and second channels of the two or more channels are separated by a rib between the first and second channels and extending along the length of the substrate, and wherein the mesh material is affixed to the rib in at least an intermittent manner along the length of the substrate.

6. The harborage of claim 5, wherein the harborage comprises a plurality of openings in the rib, said openings being sized to allow target insects to move through the openings.

7. The harborage of claim 1, wherein the substrate, when configured for growing and harvesting insects, comprises two or more channels extending diagonally along and across the substrate, and wherein each of the two or more channels is divided into a plurality of cells.

8. The harborage of claim 1, further comprising additional absorbent harborage material disposed in and along the at least one channel, the additional harborage material comprising one of or more of:
shredded material;
folded material;
baffled material;
segmented material;
crumpled material;
die-cut material; and
honeycomb material.

9. A harborage producing system, comprising:
the harborage of claim 1, disposed along a conveyor system; and
a plurality of insects disposed in and along the at least one channel of the harborage, within the confines of the substrate and the mesh material.

10. The harborage producing system of claim 9, further comprising one or more rollers disposed across the harborage at a first end of a first zone extending along the harborage, wherein the one or more rollers are disposed so as to compress the harborage and thereby form a boundary preventing the live passage of insects past the first end of the first zone.

11. The harborage producing system of claim 10, further comprising one or more additional rollers disposed across the harborage at a second end of a first zone extending along the harborage, the second end of the first zone being separated from the first end of the first zone by, a first zone length, wherein the one or more additional rollers are disposed so as to compress the harborage and thereby form a boundary preventing the live passage of insects past the second end of the first zone.

12. The harborage producing system of claim 9, further comprising a freezer, the harborage and conveyor system being disposed to pass the harborage through the freezer.

13. The harborage producing system of claim 9, further comprising a cutter, grinder, and/or shredder, the harborage and conveyor system being disposed to pass the harborage into the cutter, grinder, and/or shredder.

14. A feeder for feeding insects that feed on blood or liquid meal, the feeder comprising:
first and second chassis portions that are spaced apart by a width; and
a membrane spread out by the first and second chassis portions so as to hold the blood or liquid meal in a space formed between the first and second chassis portions, wherein the membrane comprises a material penetrable by feeding appendages of the insects.

15. The feeder of claim 14, wherein the first and second chassis portions comprise first and second chassis tubes configured for passing fluid.

16. The feeder of claim 14, wherein the first and second chassis portions are comprised of a thermally-conductive material.

17. The feeder of claim 14, further comprising one or more additional elements between the first and second chassis portions, the one or more additional elements being arranged to pass through or contact the blood or liquid meal and being configured to warm the blood or liquid meal.

18. The feeder of claim 14, further comprising at least one coupler mechanically coupled to the first and second chassis portions and configured to adjust the width between the first and second chassis portions, thereby adjusting a tightness of the membrane between the first and second chassis portions.

19. The feeder of claim 14, wherein the membrane is wrapped around the first and second chassis portions to form a membrane tube, and wherein the space that holds the blood or liquid meal is located within the membrane tube and between the chassis portions.

20. The feeder of claim 19, wherein the membrane tube is open on at least one end so as to allow a passage of the blood or liquid meal into or out of the membrane tube.

21. The feeder of claim 19, wherein at least one end of each of the first and second chassis portions is angled upwards relative to a bottom surface of the membrane tube so as to cause the blood or liquid meal to pool in the space between the first and second portions within the membrane tube and to prevent the blood or liquid meal from spilling out of the membrane tube.

22. The feeder of claim 19, further comprising a feeder insert configured to be positioned against an internal side of a top surface of the membrane tube when the membrane tube is filled with the blood or liquid meal, wherein the feeder insert is configured in size to displace a first portion of the blood or liquid meal in the membrane tube while leaving a second portion of the blood or liquid meal in the membrane tube at least between the bottom of the feeder insert and a bottom surface of the membrane tube.

23. The feeder of claim 14, wherein the membrane is a natural membrane.

24. The feeder of claim 14, wherein the membrane is a synthetic membrane.

25. A feeder for feeding insects that feed on blood or liquid meal, the feeder comprising:
a metal body comprising an internal reservoir configured for receiving a warming fluid and comprising a plurality of ribs extending along a first surface of the metal body, so as to form a plurality of channels configured to receive the blood or liquid meal; and
a membrane disposed on and affixed to the metal body on the first surface of the metal body, such that blood or liquid meal supplied to the plurality of channels is confined between the metal body and the membrane.

26. The feeder of claim 25, further comprising inlet piping and outlet piping in fluid communication with the internal reservoir.

27. The feeder of claim 25, further comprising inlet piping and exit piping in fluid communication with one or several of the plurality of chambers.
